(19) United States
(12) Patent Application Publication
(10) Pub. No.: US 2025/0040932 A1
(43) Pub. Date: Feb. 6, 2025

(54) MICROANASTOMOSIS FORCEPS

(71) Applicant: LOMA LINDA UNIVERSITY, Loma Linda, CA (US)

(72) Inventor: Miguel Angel LOPEZ-GONZALEZ, Loma Linda, CA (US)

(73) Assignee: LOMA LINDA UNIVERSITY, Loma Linda, CA (US)

(21) Appl. No.: 18/558,737

(22) PCT Filed: May 3, 2022
(86) PCT No.: PCT/US2022/027437

§ 371 Date: Nov. 2, 2023

Related U.S. Application Data
(60) Provisional application No. 63/183,458, filed on May 3, 2021.

(51) Int. Cl. A61B 17/11; A61B 17/00; A61B 17/30

(52) U.S. Cl. CPC A61B 17/11; A61B 2017/00353; A61B 2017/306

(57) ABSTRACT

A microanastomosis forceps including two longitudinally extending arms extending from a proximal pivot point. Each of the two longitudinally extending arms extends a predetermined length from the proximal pivot point to a respective distal end. A suction hose connection point adjacent to the proximal pivot point and one or more suction ports arranged on the distal end of at least one of the two longitudinally extending arms. A flow path is operably arranged between the suction hose and the one or more suction ports.

### CROSS REFERENCE TO RELATED APPLICATION

This application claims the benefit of U.S. Provisional Application No. 63/183,458, filed on May 3, 2021, which is incorporated herein by reference.

### BACKGROUND

### 1. Field of the Invention

The present inventive concept relates generally to vascular medical devices and procedures.

### 2. Description of Related Art

Microvascular anastomosis relates to a microsurgical medical procedure performed under magnification in which two tubular members (e.g. vessels, nerves, etc.) are joined together either end-to-end, end-to-side, side-to-side, or the like. Forceps are utilized within an anastomosis procedure to manipulate the vessels and/or to assist during suturing, however, forceps often have to be removed or moved within the surgical site to allow additional tools access to the procedure.

### SUMMARY

A microanastomosis forceps comprising two longitudinally extending arms extending from a proximal pivot point. Each of the two longitudinally extending arms can extend a predetermined length from the proximal pivot point to a respective distal end and a suction hose connection point can be arranged adjacent to the proximal pivot point. One or more suction ports can be arranged on the distal end of at least one of the two longitudinally extending arms and a flow path can be operably arranged between the suction hose and the one or more suction ports.

The flow path can be integrally formed through at least one of the two longitudinally extending arms. The distal end of each of the two longitudinally extending arms can include a tip. In at least one instance, the one or more suction ports can be formed on a tip of at least one of the two longitudinally extending arms.

The one or more suction ports can include a plurality of suction ports with at least one suction port on an exterior surface of the distal end of at least one of the two longitudinally extending arms and at least one suction port on the tip of at least one of the two longitudinally extending arms.

The tip of at least one of the two longitudinally extending arms can further comprise a micro-hook blunt dissecting tip extending away from an exterior surface of the distal tip. The micro-hook blunt dissecting tip can extend approximately 1 millimeter (mm) away from the exterior surface of the distal tip and has a diameter of approximately 0.75 mm.

The one or more suction ports can be arranged in a predetermined pattern along an exterior surface of the distal tip of at least one of the two longitudinally extending arms. The one or more suction ports can have a uniform cross-sectional area. The one or more suction ports can be linearly arranged along the predetermined length of at least one of the two longitudinally extending arms.

A microanastomosis forceps can include two longitudinally extending arms extending from a proximal pivot point and each of the two longitudinally extending arms can extend a predetermined length from the proximal pivot point to a respective distal end. A tip can be located at the distal end of each of the two longitudinally extending arms and can have a micro-hook blunt dissecting tip extending away from an exterior surface of the tip of at least one of the two longitudinally extending arms.

The micro-hook blunt dissecting tip can extend approximately 1 millimeter (mm) away from the exterior surface of the distal tip and has a diameter of approximately 0.75 mm.

A second micro-hook blunt dissecting tip extending away from an exterior surface of the tip of the other longitudinally extending arm. The second micro-hook blunt dissecting tip can extend approximately 1 millimeter (mm) away from the exterior surface of the distal tip and have a diameter of approximately 0.75 mm. The micro-hook blunt dissecting tip and the second micro-hook blunt dissecting tip can have different sizes, shapes, lengths, diameters, and/or profiles.

The micro-hook blunt dissecting tip can be detachably coupled with the exterior surface of the distal end. The micro-hook blunt dissecting tip can be integrally formed with the longitudinally extending arm. The two longitudinally extending arms can be formed of stainless steel and/or titanium.

### BRIEF DESCRIPTION OF THE DRAWINGS

The novel features of the disclosure are set forth with particularity in the appended claims. A better understanding of the features and advantages of the present inventive concept will be obtained by reference to the following detailed description that sets forth illustrative examples, in which the principles of the disclosure are utilized, and the accompanying drawings of which:

FIG. 1 is a diagrammatic view of dual-action microanastomosis forceps having a suction integrated therewith, according to at least one embodiment of the present inventive concept;

FIG. 2 is a detailed view of a section A-A of the dual-action microanastomosis forceps having suction integration of FIG. 1, according to at least one instance of the present disclosure;

FIG. 3 is a detailed view of a section B-B of the dual-action microanastomosis forceps having the magnified view of the suction tube embodied on one side of the forceps with additional perforations to increase the suction area of FIG. 2, according to at least one embodiment of the present inventive concept;

FIG. 4 is a diagrammatic view of dual-action microanastomosis forceps having a blunt dissector tip integrated therewith, according to at least one embodiment of the present inventive concept; and

FIG. 5 is a detailed view of a section A-A of the dual-action microanastomosis forceps having a blunt dissector tip of FIG. 4, according to at least one embodiment of the present inventive concept.

### DETAILED DESCRIPTION

Examples and various features and advantageous details thereof are explained more fully with reference to the exemplary, and therefore non-limiting, examples illustrated in the accompanying drawings and detailed in the following description. Descriptions of known starting materials and processes can be omitted so as not to unnecessarily obscure the disclosure in detail. It should be understood, however, that the detailed description and the specific examples, while indicating the preferred examples, are given by way of illustration only and not by way of limitation. Various substitutions, modifications, additions and/or rearrangements within the spirit and/or scope of the underlying inventive concept will become apparent to those skilled in the art from this disclosure.

### I. Terminology

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

Additionally, any examples or illustrations given herein are not to be regarded in any way as restrictions on, limits to, or express definitions of, any term or terms with which they are utilized. Instead, these examples or illustrations are to be regarded as being described with respect to one particular example and as illustrative only. Those of ordinary skill in the art will appreciate that any term or terms with which these examples or illustrations are utilized encompass other examples as well as implementations and adaptations thereof which can or cannot be given therewith or elsewhere in the specification and all such examples are intended to be included within the scope of that term or terms. Language designating such non-limiting examples and illustrations includes, but is not limited to: “for example,” “embodiment,” “for instance,” “e.g.,” “In some examples,” and the like.

Although the terms first, second, etc. can be used herein to describe various elements, components, regions, layers and/or sections, these elements, components, regions, layers and/or sections should not be limited by these terms. These terms are only used to distinguish one element, component, region, layer or section from another. Thus, a first element, component, region, layer or section discussed below could be termed a second element, component, region, layer or section without departing from the teachings of the present inventive concept.

### II. General Architecture

The present disclosure relates generally to a device, system, and/or method of a micro-forceps operable for implementation within a microvascular anastomosis procedure. The micro-forceps with respect to the present disclosure can have at least dual action associated therewith (e.g. suction, dissection tip, etc.) thereby allowing the micro-forceps to remain within the operating environment.

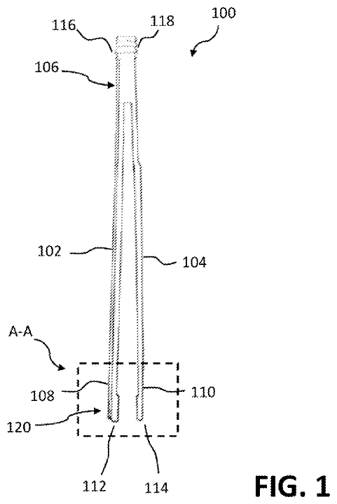

FIG. 1 illustrates a diagrammatic view of dual-action microanastomosis forceps with integrated suction, according to at least one instance of the present disclosure. The microanastomosis forceps 100 can be operably implemented within a microvascular surgical procedure in which two vessels are anastomosed together. The two vessels can be joined end to end thereby forming a substantially continuous vessel, end to side thereby forming a vessel intersection, or a side to side connection thereby forming substantially parallel vessels.

The microanastomosis forceps 100 can be operably arranged for neurosurgery environments in which magnification is implemented for precision. The microanastomosis forceps 100 can be implemented for anastomosing vessels having a diameter of approximately 1 mm using sutures (e.g. 10-0). The medical implementation can include surgeries related to cerebral ischemia often related to moyamoya disease, chronic ischemic atherosclerotic occlusive disease, and/or complex cerebral aneurysm surgeries. These surgeries often require multiple instruments within a small operating field, which can lead to remove an instrument from the operating field prior to introducing another instrument. The microanastomosis forceps 100 disclosed herein and prevent and/or eliminate the need to remove the microanastomosis forceps 100 from the operating environment to introduce additional instruments by integrating one or more instruments with the microanastomosis forceps 100. In some instances, the microanastomosis forceps 100 can be formed from any medical grade material including, but not limited to, stainless steel and/or titanium.

The microanastomosis forceps 100 can have two longitudinally extending arms 102, 104 extending from a proximal pivot point 106. Each of the two longitudinally extending arms 102, 104 can extend from the proximal pivot point 106 a predetermined distance to distal ends 108, 110, respectively. The distal ends 108, 110 of each arm 102, 104 can include a tips 112, 114, respectively. The tip 112, 114 can have a profile size and/or shape determined by the specific medical procedure being performed. In some instances, the tips 112, 114 can be blunt tipped, point tipped, rounded tip, and/or combinations thereof between the two tips 112, 114.

The proximal pivot point 106 can have a hose connection 116 adjacent thereto. The hose connection 116 can be operable to couple with a suction hose (not shown) and have a constant vacuum pressure pulled therethrough. The hose connection 116 can have a barbed 118 connection point to allow secure coupling between the suction hose and the microanastomosis forceps 100. The barbed 118 feature can allow quick coupling and de-coupling between the suction hose and the microanastomosis forceps 100 while also preventing accidental de-coupling as the microanastomosis forceps 100 are moved within the surgical environment.

The hose connection 116 can have fluidic coupling with one or more suction ports 120 formed at the distal end 108 and/or tip 112 of extending arm 102. The microanastomosis forceps 100 can have a flow path formed within the extending arm 102 providing fluidic communication between the hose connection 116 and the one or more suction ports 120. In at least one instance, the extending arm 102 has an inner bore formed therethrough providing a flow path between the hose connector 116 and the one or more suction ports 120. The flow path can allow vacuum evacuation from the one or more tips therethrough and to the hose connection 116 and the suction hose. In other instances, the extending arm 102 can have a flow path integrally formed along an exterior outer surface and/or an interior outer surface from the hose connector 116 to the distal end 108. While FIG. 1 illustrates the one or more suction ports 120 implemented with respect to extending arm 102, it is within the scope of the present disclosure to implement one or more suction ports 120 on extending arm 102, extending arm 104 and the one or more suction ports 120 specifically located at distal end 108, distal end 110, tip 112, and/or tip 114.

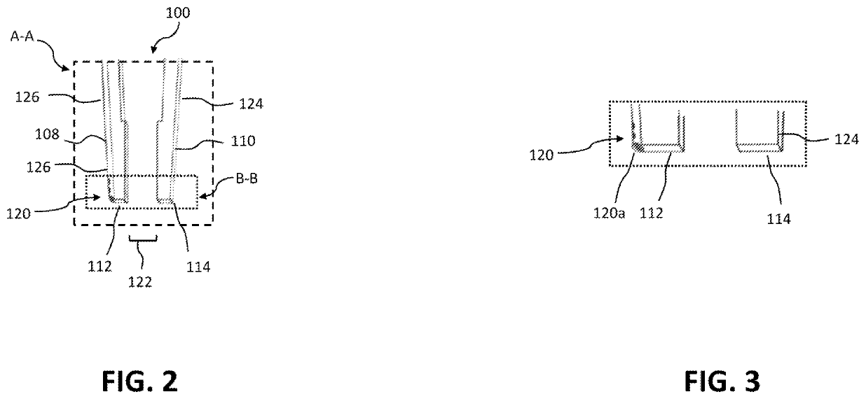

FIG. 2 illustrates a detailed view of the distal ends of the extending arms of microanastomosis forceps. The distal ends 108, 110 of the respective extending arms 102, 104 can have a predetermined spacing 122 therebetween. The predetermined spacing 122 can be operable to allow the microanastomosis forceps 100 to receive one or more anatomical and/or medical elements therein prior to being closed and/or pinched. The microanastomosis forceps 100 can be operable to articulate and/or pivot about the proximal pivot point 106, thereby allowing the extending arms 102, 104 to move closer one to the other and reduce and/or eliminate the predetermined spacing 122. A compression force can be required to articulate and/or pivot the microanastomosis forceps 100 moving the extending arms 102, 104 relative to one another about the proximal pivot point 106.

As can be appreciated in FIG. 2, the extending arm 102 can have one or more suction ports 120 disposed thereon. The one or more suction ports 120 can be arranged on the distal end 108 and be at and/or proximal to tip 112. The one or more suction ports 120 can be arranged on an exterior surface 124 of the extending arm 102. The positioning and/or location of the one or more suction ports 120 on the exterior surface 124 of the extending arm 102 can allow pivoting and/or closure of the microanastomosis forceps 100 without blocking, covering, and/or otherwise reducing the function of the one or more suction ports 120.

The microanastomosis forceps 100 can thus be implemented within the operating field without the need to remove and/or move the microanastomosis forceps 100 to introduce a new tool to the operating field. The microanastomosis forceps 100 having one or more suction ports 120 integrated therewith can allow the user and/or surgeon to keep the microanastomosis forceps 100 within the operating field during the course of the procedure.

FIG. 3 illustrates a detailed view of the one or more suction ports on a tip of a microanastomosis forceps, according to at least one instance of the present disclosure. The distal ends 108, 110 can be operably arranged to have the one or more suction ports 120 arranged thereon. The distal end 108 can include one or more suction ports 120 arranged on an exterior surface 124 thereof and/or one or more suction ports at the tip 112, thus providing suction at multiple locations and/or surfaces simultaneously. The one or more suction ports can provide a cleaning operating environment for the surgeon by removing excess fluid from the surgical site.

As can be specifically detailed in FIG. 3, the one or more suction port 120 located at the tip 112 can be proximal the exterior surface 124, thus reducing and/or eliminating any potential interference between the suction and opening and/or closing of the microanastomosis forceps 100. The flow path 126 can be integrally along the exterior surface 124 of the extended arm 102. In other instances, the flow path 126 can be integrally formed within the extended arm 102, and/or integrally formed along an interior surface of the extended arm 102 depending on the specific arrangement of the microanastomosis forceps 100.

The one or more suction ports 120 formed on the microanastomosis forceps 100 can have be an aperture formed in the exterior surface 124 of the extended arm 102, thus providing fluidic communication between ambient surgical environment, the flow path 126, and the hose connection 116 (detailed in FIG. 1). The one or more suction ports 120 can have a uniform cross-sectional area on the extended arm 102, or the one or more suction ports 120 can have varying cross-sectional areas on the extended arm 120. In at least one instance of the present disclosure, the one or more suction ports can be arranged with a plurality of suction ports 120 having a uniform cross-sectional area providing fluidic communication with the flow path 126. The plurality of suction ports 120 can be linearly arranged along a longitudinal length of the extended arm 102, arranged in a predetermined pattern, and/or arranged in any sequence or arrangement to optimize and/or improve suction within the operating environment or for the specific medical procedure.

The microanastomosis forceps 100 can have a plurality of suction ports 120 having varying cross-sectional areas to provide optimal suction in one or more desired location within the operating environment. In at least one instance of this arrangement, the plurality of suction ports can have an increasingly large cross-sectional area along the longitudinal length of the extended arm 102 with the largest of cross-sectional area being proximal to the tip 112 with the smallest cross-sectional arear being distal to the tip 112. In other instances of this arrangement, the plurality of suction ports 120 can be in any predetermined arrangement by size, shape, pattern, or the like desirable to the operating environment or for the specific medical procedure.

As can be further appreciated in FIG. 3, the plurality of suction ports 120 of the microanastomosis forceps 100 can include at least one suction port 120a operably arranged on the tip 112. The suction port 120a operably arranged at the tip 112 can provide suction in a region adjacently below the distal end 108 of the extended arm 102 while remaining suction ports 120 arranged on the exterior surface 124 of the extended arm 120 can provide suction in a region adjacent to the distal end 108 and/or the tip 112. The plurality of suction ports 120 can be formed on one or more surfaces of the distal end 108 and/or the tip 112 to provide multi-directional suction within the operating field.

While FIG. 3 illustrates a single suction port 120a formed at the tip 112, it is within the scope of the present disclosure to implement a one suction port 120a or a plurality of suction ports 120a arranged on the tip 112 in combination with and/or independent of one or more suction ports 120 arranged on the distal end 108 of the extending arm 102. Further, while FIG. 3 illustrates the one or more suction ports 120 arranged with extending arm 102, it is within the scope of the present disclosure to implement one or more suction ports on extension arm 102, extension arm 104, and/or combinations thereof. In some instances, the microanastomosis forceps 100 can have one or more suction ports 120 on extension arm 102 and extension arm 104.

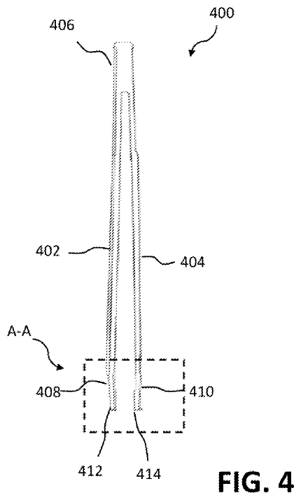

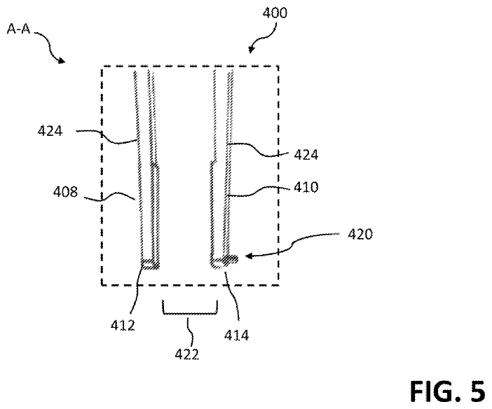

FIG. 4 illustrates a microanastomosis forceps having a blunt tip hook, according to at least one instance of the present disclosure. As discussed above with respect to FIGS. 1-3, the microanastomosis forceps 400 can be operably arranged for neurosurgery environments in which magnification is implemented for precision. The microanastomosis forceps 400 can be implemented for anastomosing vessels having a diameter of approximately 1 mm using sutures (e.g. 10-0). The medical implementation can include surgeries related to cerebral ischemia often related to moyamoya disease, chronic ischemic atherosclerotic occlusive disease, and/or complex cerebral aneurysm surgeries. These surgeries often require multiple instruments within a small operating field, which can lead to remove an instrument from the operating field prior to introducing another instrument. The microanastomosis forceps 400 disclosed herein and prevent and/or eliminate the need to remove the microanastomosis forceps 400 from the operating environment to introduce additional instruments by integrating one or more instruments with the microanastomosis forceps 400. In some instances, the microanastomosis forceps 400 can be formed from any medical grade material including, but not limited to, stainless steel and/or titanium.

The microanastomosis forceps 400 can have two longitudinally extending arms 402, 404 extending from a proximal pivot point 406. Each of the two longitudinally extending arms 402, 404 can extend from the proximal pivot point 406 a predetermined distance to a distal end 408, 410. The distal end 408, 410 of each arm 402, 404 can include a tips 412, 414, respectively. The tip 412, 414 can have a profile size and/or shape determined by the specific medical procedure being performed. In some instances, the tips 412, 414 can be blunt tipped, point tipped, rounded tip, and/or combinations thereof between the two tips 412, 414.

The microanastomosis forceps 400 can have a predetermined spacing 422 between the distal ends 408, 410 of the respective extending arms 402, 404. The predetermined spacing 422 can be operable to allow the microanastomosis forceps 400 to receive one or more anatomical and/or medical elements therein prior to being closed and/or pinched. Specifically, in a microvascular anastomosis procedure, the microanastomosis forceps 400 can be implemented for adjusting and/or manipulating one or more vessels before, during, and/or after suturing, thus requiring the articulation of the microanastomosis forceps 400 by adjusting the predetermined spacing between the distal ends 408, 410 through pivoting movement of the extending arms 402, 404, respectively, about the proximal pivot point 406.

The microanastomosis forceps 400 can be operable to articulate and/or pivot about the proximal pivot point 106, thereby allowing the extending arms 402, 404 to move closer one to the other and reduce and/or eliminate the predetermined spacing 422. A compression force can be required to articulate and/or pivot the microanastomosis forceps 400 moving the extending arms 402, 404 relative to one another about the proximal pivot point 406.

FIG. 5 illustrates a detailed view of section A-A of the microanastomosis forceps of FIG. 4. The tips 412, 414, respectively of the extending arms 402, 404 can be coupled with one or more blunt hook dissector tip 420 attachments. The one or more blunt hook dissector tip 420 attachments can be operable to assist in opening and/or displacing a flap or associated patient tissue from either a recipient vessel and/or a donor vessel during the course of a microvascular anastomosis procedure.

As can be appreciated in FIG. 5, a micro-hook blunt dissector tip 420 can be attached to the distal end 410 of extension arm 404 substantially proximal to the tip 422. The blunt hook dissector tip 420 can be a blunt tip protrusion away from an exterior surface 424 of extending arm 404. The size and/or shape of the blunt hook tip protrusion can be determined by the specific medical procedure being performed and/or the size of the vessels implemented within the anastomosis procedure. In at least one instance of the present disclosure, the blunt hook tip 420 can be approximately 1 millimeter (mm) in length with a diameter of 0.75 mm. The length of the blunt hook tip 420 can be defined as the protruding distance away from the exterior surface 424 of extending arm 404. The 0.75 mm diameter blunt hook tip 420 can be operably implemented for micro-vascular anastomosis procedures involving vessels of approximately 1.0 mm diameter. In other instances, the blunt hook dissector tip 420 can be sized and/or shaped in view of the medical procedure and/or surgeon preference.

The blunt hook dissector tip 420 operably disposed at the distal end 410 of extending arm 404 can prevent a surgeon or other medical professional from being required to remove and/or move the microanastomosis forceps 400 from the operating environment to be able to introduce a micro-blunt tip hook. The microanastomosis forceps 400 as detailed in FIGS. 4 and 5 can have a micro-blunt tip hook 420 integrated therewith allowing the microanastomosis forceps 400 to remain within the operating environment throughout the medical procedure.

While FIGS. 4 and 5 detail a micro-hook blunt tip 420 integrated with distal end 410 and extending arm 404, it is within the scope of the present disclosure to implement a micro-hook blunt tip 420 with extending arm 402 and/or extending arm 404. In at least one instance, the microanastomosis forceps 400 can implement a micro-hook blunt tip 420 on each extending arm 402, 404, respectively. In some instances, the microanastomosis forceps 400 can implement two micro-hook blunt tips 420 with a micro-hook blunt tip 420 on each distal end 408, 410 in which each of the two micro-hook blunt tips 420 have different sizes, shapes, lengths, diameters, and/or profiles, thus providing flexibility for the operator within the operating environment. In yet other instances, the microanastomosis forceps 400 can implement two micro-hook blunt tips 420 on each distal end 408, 410 in which each of the two micro-hook blunt tips 420 have the same size, shape, length, diameter, and/or profile, thus preventing a user from having to re-orientate the microanastomosis forceps 400 within the operating environment as needed.

Further, the micro-hook blunt dissector tip 420 can be removable in medical procedures in which it is unnecessary and/or may interfere within the operating environment. In other instances, the micro-hook blunt dissector tip 420 can be modular to allow removing, interchanging, and/or replacing the micro-hook blunt dissector tip 420. This can provide the opportunity to insure the appropriate micro-hook blunt dissector tip 420 size, shape, length, diameter, and/or profile for the medical procedure and/or replacement of a worn and/or damaged micro-hook blunt dissector tip 420 without requiring replacement of the microanastomosis forceps 400.

In some instances of the present disclosure, one or more features of the microanastomosis forceps 100, 400 as detailed in FIGS. 1-5 can be modular, adjustable, and/or interchangeable depending on the medical procedure, the operating environment, patient information, and/or surgeon preference.

It is within the scope of the present disclosure to implement a microanastomosis forceps with an extending arm 102, 104 having a micro-hook blunt dissector tip 420 arranged thereon and/or an extending arm 402, 404 having one or more suction ports 120 arranged thereon and coupled with a flow path and a hose connector. Further, it is within the scope of this disclosure that one or more features can be modular and added/removed in preparation for a medical procedure.

While preferred examples of the present inventive concept have been shown and described herein, it will be obvious to those skilled in the art that such examples are illustrative only. Further, it is within the scope of the present disclosure to combine one or more features of the illustrative examples disclose herein without deviating from the present disclosure. Numerous variations, changes, and substitutions will now occur to those skilled in the art without departing from the disclosure. It should be understood that various alternatives to the examples of the disclosure described herein can be employed in practicing the disclosure. It is intended that the following claims define the scope of the disclosure and that methods and structures within the scope of these claims and their equivalents be covered thereby.

What is claimed is:

1. A microanastomosis forceps, the forceps comprising:
two longitudinally extending arms extending from a proximal pivot point, each of the two longitudinally extending arms extend a predetermined length from the proximal pivot point to a respective distal end; and
a suction hose connection point adjacent to the proximal pivot point;
one or more suction ports arranged on the distal end of at least one of the two longitudinally extending arms;
wherein a flow path is operably arranged between the suction hose and the one or more suction ports.

2. The microanastomosis forceps of claim 1, wherein the flow path is integrally formed through at least one of the two longitudinally extending arms.

3. The microanastomosis forceps of claim 1, wherein the distal end of each of the two longitudinally extending arms includes a tip.

4. The microanastomosis forceps of claim 3, wherein the one or more suction ports are formed on a tip of at least one of the two longitudinally extending arms.

5. The microanastomosis forceps of claim 3, wherein the one or more suction ports includes a plurality of suction ports with at least one suction port on an exterior surface of the distal end of at least one of the two longitudinally extending arms and at least one suction port on the tip of at least one of the two longitudinally extending arms.

6. The microanastomosis forceps of claim 3, wherein the tip of at least one of the two longitudinally extending arms further comprises a micro-hook blunt dissecting tip extending away from an exterior surface of the distal tip.

7. The microanastomosis forceps of claim 6, wherein the micro-hook blunt dissecting tip extends approximately 1 millimeter (mm) away from the exterior surface of the distal tip and has a diameter of approximately 0.75 mm.

8. The microanastomosis forceps of claim 1, wherein the one or more suction ports are arranged in a predetermined pattern along an exterior surface of the distal tip of at least one of the two longitudinally extending arms.

9. The microanastomosis forceps of claim 1, wherein the one or more suction ports have a uniform cross-sectional area.

10. The microanastomosis forceps of claim 1, wherein the one or more suction ports are linearly arranged along the predetermined length of at least one of the two longitudinally extending arms.

11. A microanastomosis forceps, the forceps comprising:
two longitudinally extending arms extending from a proximal pivot point, each of the two longitudinally extending arms extend a predetermined length from the proximal pivot point to a respective distal end;
a tip located at the distal end of each of the two longitudinally extending arms; and
a micro-hook blunt dissecting tip extending away from an exterior surface of the tip of at least one of the two longitudinally extending arms.

12. The microanastomosis forceps of claim 11, wherein the micro-hook blunt dissecting tip extends approximately 1 millimeter (mm) away from the exterior surface of the distal tip and has a diameter of approximately 0.75 mm.

13. The microanastomosis forceps of claim 11, further comprising a second micro-hook blunt dissecting tip extending away from an exterior surface of the tip of the other longitudinally extending arm.

14. The microanastomosis forceps of claim 13, wherein the second micro-hook blunt dissecting tip extends approximately 1 millimeter (mm) away from the exterior surface of the distal tip and has a diameter of approximately 0.75 mm.

15. The microanastomosis forceps of claim 13, wherein the micro-hook blunt dissecting tip and the second micro-hook blunt dissecting tip have different sizes, shapes, lengths, diameters, and/or profiles.

16. The microanastomosis forceps of claim 11, wherein the micro-hook blunt dissecting tip is detachably coupled with the exterior surface of the distal end.

17. The microanastomosis forceps of claim 11, wherein the micro-hook blunt dissecting tip is integrally formed with the longitudinally extending arm.

18. The microanastomosis forceps of claim 11, wherein the two longitudinally extending arms are formed of stainless steel and/or titanium.
